(19) United States
(12) Patent Application Publication
(10) Pub. No.: US 2012/0135376 A1
(43) Pub. Date: May 31, 2012

(54) Collagen Biomaterial Wedge

(75) Inventors: David Cheung, Arcadia, CA (US); Edwin Shors, Laguna Beach, CA (US); William Knox, Newport Beach, CA (US)

(73) Assignee: Osseous Technologies of America, Newport Beach, CA (US)

(21) Appl. No.: 13/262,541

(22) PCT Filed: Mar. 30, 2010
(86) PCT No.: PCT/US10/29166

§ 371 Date: Feb. 13, 2012

Related U.S. Application Data
(60) Provisional application No. 61/164,623, filed on Mar. 30, 2009.

(51) Int. Cl. A61C 19/06; B29C 39/38; A61P 43/00; A61C 5/14; A61K 9/00; A61K 38/39

(52) U.S. Cl. 433/136; 424/400; 514/17.2; 433/215; 264/28

(57) ABSTRACT

A biocompatible, resorbable collagen membrane having a wedge shape with a thick edge of relatively higher strength and rigidity and a thin edge of relatively higher deformability and elasticity, which membrane is bendable to a desired configuration and is sufficiently rigid to retain the bent configuration upon implantation at a surgical site; a method of making such a membrane, and the use of such a membrane in a “sinus lift” procedure for augmenting alveolar bone.

### FIELD OF THE INVENTION

The present invention relates to a malleable collagen membrane for guided tissue regeneration in a human or other mammal.

### BACKGROUND OF THE INVENTION

Bone is the body's primarily structural tissue; consequently it can fracture and biomechanically fail. Fortunately, it has a remarkable ability to regenerate because bone tissue contains stem cells which are stimulated to form new bone within bone tissue and adjacent to the existing bone. Boney defects regenerate from stem cells residing in viable bone, stimulated by signally proteins, and multiplying on existing cells or on an extracellular matrix (i.e., trellis). Like all tissues, bone requires support via the vascular system to supply nutrients and cells, and to remove waste. Bone will not regenerate without prompt regeneration of new blood vessels (i.e., neovascularization), typically with the first days and weeks of the regenerative cascade.

Various attempts have been made in the past to stimulate or augment bone regeneration by introducing a bone regenerating material proximate to a deteriorated bone structure. Such efforts have met with very limited success, however, because they have not been able adequately to control the placement of the bone regenerating material and thus guide the development of new or additional bone. Measures undertaken to control the placement of the bone regenerating material may hinder cell ingrowth and formation of blood vessels needed for development of additional bone and thus impede the desired bone regeneration. Thus, despite considerable efforts of the prior art, there has remained a long felt need for better methods of tissue augmentation, especially for bone regeneration or augmentation.

A major problem encountered by dentists, particularly oral surgeons and periodontists, is restoration or regeneration of the edentulous maxilla. Due to atrophy of the alveolar ridge and enlargement of the maxillary sinus, particularly after tooth loss, the maxilla often becomes a thin layer of bone. To restore function and cosmetics, dental implants are inserted into the maxilla. However, dental implants require sufficient bone engagement with their metal surface to biologically anchor them into the maxilla. This biological process is called osteointegration. If the maxilla is insufficiently thick to support dental implants, the surgeon, therefore, may regenerate bone within the maxillary sinus to provide adequate osteointegration of the dental implant.

A particular problem can arise when an oral or maxillofacial surgeon seeking to augment the bone of the alveolar ridge undertakes what is known as a sinus lift procedure as described, for example, in published European patent application no. EP 1 174 094 A1. In this procedure, the sinus cavity is penetrated through a buccal window incision and the Schneiderian membrane is released and reflected superiorly to provide a cavity for introduction of bone graft material. The Schneiderian membrane is problematic for the surgeon because it is thin, compliant and fragile. The Schneiderian membrane is attached to the bone of the maxillary sinus. It can be detached using either surgical hand tools or by inserting a balloon catheter into a tunnel and inflating the balloon. The balloon catheter more gently separates the membrane from the bone. Not infrequently however, the Schneiderian membrane becomes torn and requires repair. Otherwise, bone graft material introduced into the cavity formed by lifting the Schneiderian membrane can leak into the sinus through the tear.

Materials heretofore used to repair torn Schneiderian membranes have typically been made of highly porous collagen. Collagen has been used as an implantable biomaterial for more than 50 years. The collagen used for biomedical implants is either derived from animals (e.g., cows, pigs, horses) and humans, or it is manufactured in vitro using recombinant engineering. It is known to be biocompatible and is resorbed and remodeled like natural tissues, via cellular and enzymatic processes.

Conventional highly porous implantable collagen membranes typically have been made of reconstituted, reticulated bovine (i.e., cow) collagen. Such materials are conventionally provided to surgeons as rectilinear sheets with uniform thicknesses of approximately 1 mm. Their low density and high porosity make such materials supple and conformable. Unfortunately, however, they therefore also have a low tensile strength and stiffness, particularly after wetting with saline or blood, and are inadequate for use as a containment device in surgical applications. Rather, they are difficult to handle and liable to tear themselves. In addition, such materials are difficult to retain in a desired position because they are so thin and fragile that they are difficult to attach at the desired location with a bone tack or suture.

### SUMMARY OF THE INVENTION

The present invention provides a malleable, wedge-shaped sheet or membrane of resorbable collagen which may be used by surgeons as an implantable medical device to aid in a variety of tissue regenerative indications. Heretofore, sheets or membranes of collagen have been either highly porous and biomechanically weak or they have been minimally porous and biomechanically strong. For many tissue regenerative indications, it is desirable to have the sheets or membranes of collagen with areas of high strength and stiffness, and at the same time with other areas of high porosity. High strength and stiff collagen provides structure for containing or retaining cells, growth factors or particulate matrices; however low porosity precludes the ingrowth of blood vessels and regenerative cells. Highly porous collagen permits essential ingrowth but does not contain or retain cells, growth factors or particulate matrices at a targeted location.

The present invention provides a resorbable biomaterial for guided tissue regeneration which is wedge-shaped, with a thicker area designed for high strength and a thinner area designed for optimum formability. This wedged shaped membrane is strong, tough and malleable. The invention thus provides a biocompatible and resorbable collagen membrane, for guided tissue regeneration which is ideal for many bone reconstructive indications.

The wedge-shaped collagen membranes of the invention serve three functions. First, they serve as a protective barrier that may prevent penetration of the Schneiderian Membrane intra-operatively. Secondly, they serve as a trellis for tissue regeneration, particularly promoting regeneration of fibrovascular tissue to reinforce the Schneiderian Membrane if there is a tear or potential tear. The collagen is biocompatible and porous for ingrowth of connective tissue. Third, they serve as a biocompatible structural barrier, allowing the clinician to more easily visualize the space within the maxillary sinus prior to placing bone graft materials and assisting in containing biomaterials at a desired location and/or in a desired configuration.

Trellises of porous biomaterials (i.e., matrices) serve as a framework on which and through which tissue can grow. Most tissues, including bone and fibrovascular tissue, proliferate only by attaching to a structure or matrix. Cells then multiply and expand on pre-existing cells, extra-cellular matrix or biomaterials. Therefore, these matrices must have porosity. However, porosity generally decreases strength, typically non-linearly such that a small amount of porosity disproportionally decreases mechanical properties. The optimal porosity has been characterized in the musculoskeletal, field, for various principal regenerative tissues. For neovascular tissue (i.e., new blood vessels), pore diameters must be larger than 20 micrometers. For osteoid (non-mineralized bone), pore diameters must be larger than 50 micrometers. For bone formation, pore diameters must be larger than 100 micrometers.

Tissue regeneration is a race between competing tissues. Whichever tissue fills the space first, will dominate. Fibrovascular tissues ordinarily proliferate faster than bone tissue. Consequently, fibrovascular tissue may preferentially fill in a defect where bone is desired, resulting in scar tissue.

Assuring precise positioning of implanted tissue augmentation materials in a living body can be a difficult task. Moreover, because a living body is a dynamic environment, implanted materials may shift in position over time. The use of strategically shaped and implanted membranes according to the present invention, however, facilitates precise placement of implanted biomaterials and enables containment or retention of the implanted biomaterial at the desired location within the body.

The present invention makes use of collagen as a resorbable biomaterial for implantable medical devices to aid in tissue regeneration and repair. Depending on the extent of cross linking, collagen biomaterials can be manufactured to resorb over a prescribed range, typically from a few weeks to one year.

The present invention uses collagen membranes having a wedge shape to facilitate tissue regeneration, particularly bone and fibrovascular tissue. This wedge shape can be manufactured by casting collagen between mold plates which form a wedge shape between them and lyophilizing, to form a highly porous structure. The resulting wedge-shaped collagen membranes are then moistened and dried. This process increases the density and cross linking to provide high strength, strong, stiff membranes which are nevertheless sufficiently malleable to be formed into a desired configuration to fit a surgical site in order to support a tissue membrane and/or retain surgically introduced bone graft material in a desired location.

The wedge-shaped collagen membranes of the invention can be manufactured by a casting process using mold plates which form a V-shaped mold cavity between them. The mold cavity is filled with a collagen suspension. After lyophilization, the mold is opened and the resulting wedge-shaped membrane removed. The membrane can then be rehydrated and dried to provide a high strength three dimensional form.

If desired, macroscopic holes can be made in the membrane with strategically placed pins transecting the mold cavity which are removed before the mold is opened. Alternatively, macroscopic holes can then be made in the membrane after rehydration and drying with strategically placed pins, cuts, or laser cutting. In yet another alternative, the membrane may be made by a selective rehydration/drying process in which a selected portion of the membrane is rehydrated and dried to provide a high strength three dimensional form while the remaining portion that is not rehydrated/dried retains an open porosity, but has a lower strength and stiffness.

The wedge-shaped collagen membrane of the invention has a number of important advantages for guided tissue regeneration. The thinner portion of the membrane exhibits optimal porosity to assure neovascular ingrowth and bone cell ingrowth because pores of the required dimensions are precisely manufactured.

The wedge-shaped collagen membrane of the invention also exhibits optimal strength. The membrane of the invention assures that the optimal mechanical properties are provided in collagen membranes so that they can be formed by bending and/or cutting to a desired configuration to match an intended surgical site and afterward will retain that configuration under normal loading conditions.

The thicker edge of the wedge-shaped membrane improves user-friendliness for the surgeon by making it easier for the surgeon to identify the proper orientation of the membrane and also by facilitating handling.

The thicker edge of the wedge-shaped membrane also provides a site at which the membrane can be tacked to existing bone adjacent the surgical site, (e.g., at the external buccal wall) with one or more bone tacks or sutures to retain it in a desired position. Because the thicker edge exhibits stronger mechanical properties, such as tensile strength or tear strength, due to its larger cross-sectional area, the wedge-shaped membrane exhibits greatly improved resistance to tearing when a bone tack or suture is placed through the membrane. Also the thick edge stabilizes bone tacks in the collagen to make it easier for the surgeon to identify pre-drilled bone holes or to tap the tacks into the bone.

The thickness of the thick edge may range from about 1 mm to about 5 mm, preferably about 1.5 mm to about 3.5 mm, and particularly preferably about 2 mm. The transition between the thick edge and the thin edge may be linear, or in other words, the wedge-shaped membrane may have a uniform taper from the thick edge to the thin edge, thereby giving rise to a smooth surface. Alternatively, the transition between the thick edge and the thin edge may be a step function, giving rise to a membrane comprised of adjacent sections each having a progressively smaller thickness.

The thin portion of the wedge-shaped membrane provides a collagen membrane that is simultaneously both malleable and resiliently elastic.

By malleable is meant that the membrane can be folded to a desired shape or configuration and then will retain that configuration. This is achieved by bending the membrane beyond the elastic limit of the material and then creasing the membrane at the bending site. As a result, the membrane will retain its shape after being custom bent, intra-operatively by the surgeon.

By resiliently elastic is meant that the membrane is semi-rigid but will readily deform when pressed into contact with the surgical site so as to conform to the configuration of the surgical site. At the same time it resists permanent shape change so that restoring forces in the membrane will urge the membrane to reassume its original configuration, thereby biasing the membrane against the surgical site. This is achieved insofar as the elastic limit of the membrane is not exceeded so that no permanent deformation arises.

The thickness of the thin edge may range from about 0.3 mm to about 1.5 mm, preferably from about 0.4 mm to about 1.0 mm, and particularly preferably about 0.5 mm.

The thin edge may also be easily trimmed by scissors or scalpel to fit the surgical site. It is preferred to trim the membrane to slightly oversize dimensions so that a snug fit will be generated due to the resilient elasticity of the membrane.

This combination of malleability and resilient elasticity results in a membrane which is readily formable and bendable by the surgeon to fit the surgical site and which provides a snug fit to assure positional stability of the membrane and also effective retention of bone graft material in the desired location.

The combination of ease of handling provided by the thicker edge of the wedge-shaped membrane and the ease of fit provided by the thinner edge of the membrane also provides convenience for the surgeon who uses it. In addition, operating time by the surgeon and staff is conserved by using the wedge-shaped membranes of the invention. The wedge shape of the membrane and the mechanical properties of the invention also have the advantage that infection rates are decreased because excessive handling of the biomaterial and excessive shaping/cutting time is eliminated.

As used herein, the term “lyophilization” refers to “freeze drying” or vacuum drying.

In the process for producing the membranes of the invention, the molded collagen suspension is placed in a freezer and then a vacuum is applied. Under vacuum, the water within the collagen moves directly from the solid phase to the gas phase. Consequently, there is no shrinking or change to the dimensions. This makes a highly porous, but relatively weak collagen structure. A key step in the production process according to the invention is then to lightly wet the porous collagen with alcohol/water, which collapses the porosity. The material is then air dried. This makes a much stronger/stiffer collagen membrane. Air drying at elevated temperatures also cross-links some of the collagen molecules to further increase the strength and decrease the resorption rate.

### BRIEF DESCRIPTION OF THE FIGURES

The invention will be described in further detail hereinafter with reference to an illustrative example of a preferred embodiment shown in the accompanying figures, in which:

FIG. 1 is a perspective view of an illustrative collagen biomaterial wedge in accordance with the present invention; and

FIGS. 2a through 2d are successive sectional views showing the use of a collagen biomaterial wedge according to the invention to support and repair a torn Schneiderian membrane in the course of a sinus lift procedure.

### DETAILED DESCRIPTION OF THE INVENTION

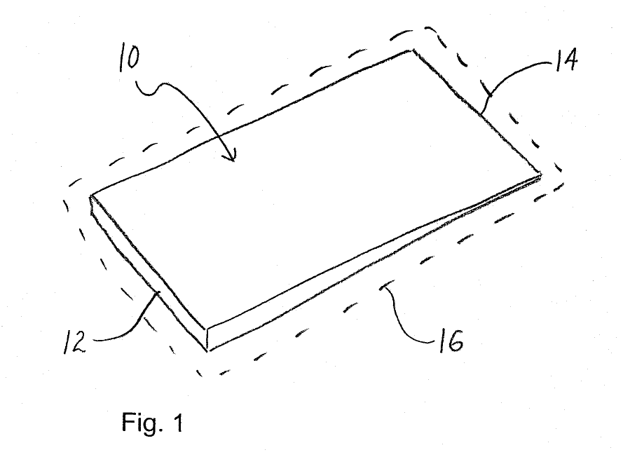

FIG. 1 is a perspective view of a wedge-shaped, densified collagen biomaterial membrane according to the invention. As shown in FIG. 1, membrane 10 has a generally rectangular configuration, but it should be understood that the membrane could as well have an oval or generally triangular configuration. Membrane 10 has a thicker edge 12, which provides increased strength for handling and/or for attachment to a bone adjacent the surgical site with one or more bone tacks in order to hold the membrane in the desired position. Membrane 10 tapers gradually to a thinner edge 14, which provides increased deformability in order to facilitate proper mating with the configuration of the surgical site. The membrane can be easily trimmed during surgery with scissors or a scalpel for a custom fit to the surgical site. The membrane need not be wetted prior to implantation, but can be wetted in place with saline or blood from the surgical site. Membrane 10 can be bent to a desired configuration to fit the surgical site and generally has sufficient rigidity to retain the desired configuration so that it can retain implanted bone graft material in the desired location. At the same time, especially the thinner edge 14 of membrane 10 is sufficiently supple and resiliently elastic that it will conform to the configuration of the surgical site without excessive trimming or shaping and can be quickly placed by the surgeon.

Collagen membrane is preferably distributed in a sterile package, which is depicted schematically in FIG. 1 by broken line 16.

The wedge-shaped collagen biomaterial membrane of the invention can be produced as follows. A suspension of purified collagen is made in water/alcohol. The collagen is preferably in native fibrous form with a fiber length of from about 0.2 to 3 mm, preferably about 1.5 mm. The suspension advantageously may contain from about 10 to about 60 mg of collagen per ml of suspension, particularly preferably from about 15 to about 20 mg collagen per ml. The suspending medium may advantageously comprise from about 5% to about 25% ethanol in water, particularly preferably about 10% ethanol.

After deaeration of the collagen suspension, the suspension is filled into a mold made up of two mold plates inserted into vertical V-shaped slots on the end plates of a main frame so that the plates form a V-shaped mold cavity. The filled mold is then placed in a freezer at a temperature sufficient to solidify the suspension, e.g., −70° C. Once the suspension is solidified, the plates are separated, with the frozen collagen wedge remaining on one of the plates.

The mold plate with the collagen wedge is then transferred to a freeze dryer and freeze dried. The freeze-dried collagen wedge is then removed from the freeze dryer. The dried collagen is sprayed with an alcohol solution. Preferably the alcohol solution may contain about 40 to about 70% alcohol in water, particularly preferably about 50% ethanol in water. The wedge-shaped membrane is then subject to air drying followed by vacuum drying until completely dry. Thereafter, the dried wedge-shaped membrane is subjected to heat treatment at from about 100 to about 140° C. for from about 15 minutes to about 2 hours to cure the membrane. Particularly preferably the membrane is cured for about one-half hour at a temperature of approximately 130° C.

After curing, the membrane may be cut to desired size and sterilely packaged for distribution and use.

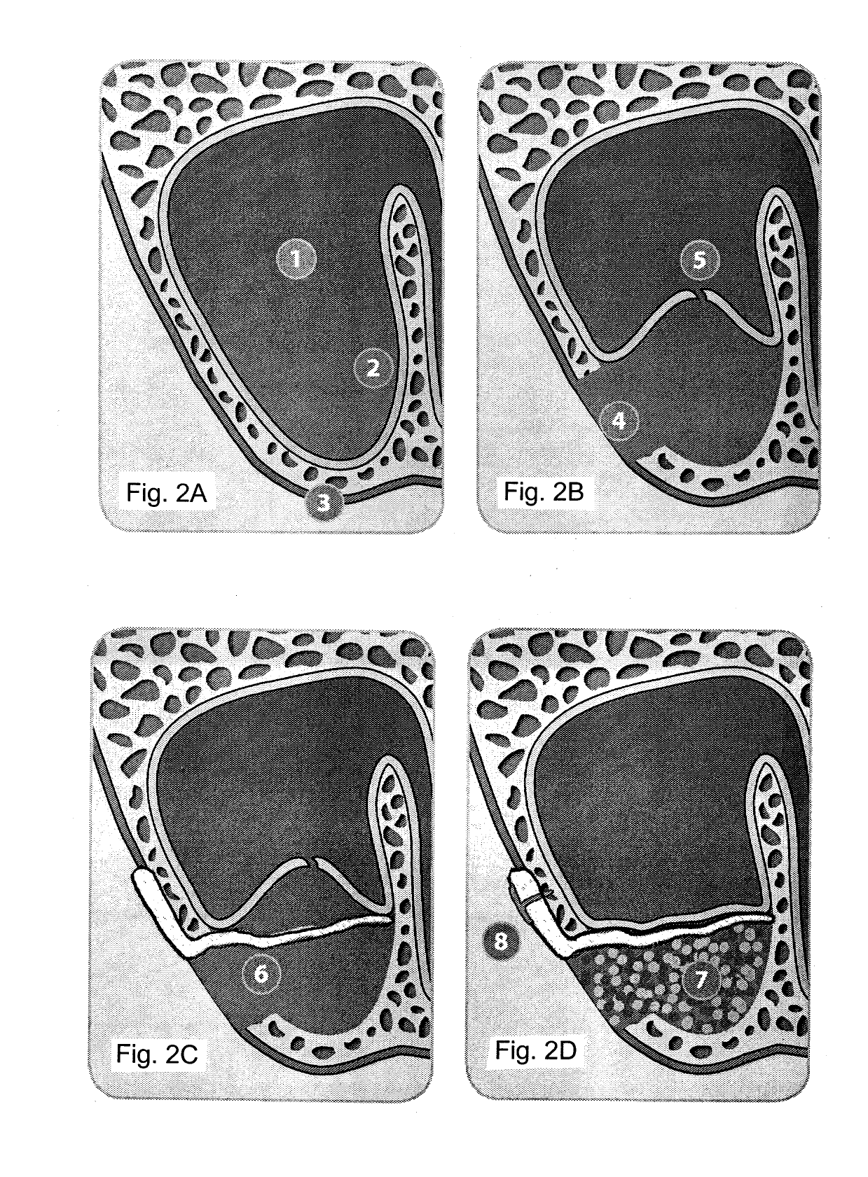

FIGS. 2A through 2D show an example of the use of the wedge shaped collagen membrane of the invention.

FIG. 2A is a sectional view thorough a sinus cavity 1 with the Schneiderian membrane 2 separating the sinus from the alveolar ridge 3. In FIG. 2B a lateral osteotomy 4 has been made through the buccal wall, and the Schneiderian membrane 2 has been released and elevated, e.g. using an inflatable balloon. As a result, a slight tear 5 has formed in the fragile Schneiderian membrane.

In FIG. 2C, an appropriately cut and shaped, wedge-shaped collagen membrane 6 has been inserted into the incision so as to underly and support repair of the torn Schneiderian membrane. Membrane 6 is custom bent by the surgeon into an L-configuration with the thicker end lying alongside the buccal wall and the thinner end extending across the sinus cavity to the opposite wall. As evidenced by the slight undulation in the membrane 6, the thin edge of the membrane is pressed tightly against the sinus wall sufficient to slightly deform the membrane and assure a tight fit. Despite wetting by saline or blood from the incision, the collagen membrane remains sufficiently semi-rigid to retain its customized shape and position.

FIG. 2D shows the thicker edge of the membrane 6 secured to the buccal wall with a bone tack 8. A more stable attachment can be achieved because of the greater thickness and consequent strength of the thicker edge of the collagen membrane 6 which greatly decreases the possibility that the collagen membrane will tear free of the bone tack. The cavity formed by elevation of the sinus membrane is filled with bone graft material 7 to augment the alveolar ridge and provide sufficient bone depth for implantation of a dental implant. Collagen membrane 6 serves both to retain the bone implant material in the desired location and to form a new sinus floor to support the Schneiderian membrane while the membrane heals. The collagen membrane actually tents up the Schneiderian membrane to prevent compression of bone graft material which lacks structure such as bone morphogenic protein (BMP) in collagen sponge. Due to its biologic character, the collagen membrane is eventually resorbed.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A biocompatible, resorbable collagen membrane having a wedge shape with a thick edge of relatively higher strength and rigidity and a thin edge of relatively higher deformability and elasticity, wherein said membrane is bendable to a desired configuration and is sufficiently rigid to retain the bent configuration upon implantation to a surgical site.

2. A membrane as claimed in claim 1, wherein said membrane is bent to an L-shaped configuration.

3. A membrane as claimed in claim 1, wherein said membrane is enclosed in a sterile package.

4. A membrane as claimed in claim 1, wherein said thick edge is stably tacked to bone adjacent the surgical site by at least one bone tack.

5. A membrane as claimed in claim 1, wherein said membrane has a uniform taper from the thick edge to the thin edge.

6. A membrane as claimed in claim 1, wherein said thick edge has a thickness of about 1 to about 5 mm, and said thin edge has a thickness of about 0.3 to about 1.5 mm.

7. A membrane as claimed in claim 6, wherein said thick edge has a thickness of about 2 mm, and said thin edge has a thickness of about 0.5 mm.

8. A method of making a biocompatible, resorbable collagen membrane, said method comprising:
forming a suspension of collagen fibers;
filling the suspension into a wedge-shaped mold;
freezing the filled mold to solidify the suspension into a wedge-shaped member;
thereafter freeze-drying the wedge-shaped member;
spraying the freeze-dried wedge-shaped member with a water/alcohol solution;
vacuum drying the wedge-shaped member; and
heat treating the vacuum-dried member.

9. A method as claimed in claim 8, wherein said wedge-shaped member is air dried after the spraying and before the vacuum drying.

10. A method as claimed in claim 8, wherein said suspension comprises collagen fibers having a fiber length from about 0.2 to about 3 mm suspended in a water/alcohol suspending agent comprising from about 5 to about 25% alcohol and contains from about 10 to about 60 milligrams of collagen fibers per milliliter of said suspension.

11. A method as claimed in claim 10, wherein said suspension comprises about 15 mg/ml collagen fibers having an average fiber length of about 1.5 mm suspended in a ethanol/water suspending agent comprising about 10% ethanol.

12. A method as claimed in claim 8, wherein said freezing is effected at a temperature of about −70° C. or lower.

13. A method as claimed in claim 8, wherein said spraying is effected with a alcohol/water solution comprising from about 40 to about 70% alcohol.

14. A method as claimed in claim 13, wherein said alcohol/water solution comprises about 50% ethanol.

15. A method as claimed in claim 8, wherein said heat treating is effected at a temperature of from about 100 to about 140° C. for a time from about 15 minutes to about 2 hours.

16. A method as claimed in claim 15, wherein said heat treating is effected at a temperature of about 130° C. for about 30 minutes.

17. A method of augmenting alveolar bone in a patient, said method comprising:
forming a lateral osteotomy through the buccal wall into a lower portion of a sinus cavity;
inserting an inflatable balloon into the lateral osteotomy and inflating the balloon to release and elevate the Schneiderian membrane of the sinus cavity, thereby forming a space under the Schneiderian membrane;
disposing the thinner end of a wedge-shaped, biocompatible and resorbable collagen membrane underneath the elevated Schneiderian membrane;
securing the thicker end of the wedge-shaped collagen membrane to the buccal wall; and
filling the space underneath the Schneiderian membrane with bone graft material;
whereby said wedge-shaped collagen membrane retains said bone graft material in position.
